(19) United States
(12) Patent Application Publication
(10) Pub. No.: US 2018/0289867 A1
(43) Pub. Date: Oct. 11, 2018

(54) POLYETHERIMIDE COMPOSITIONS FOR IMPLANTABLE MEDICAL DEVICES AND SPACERS THEREOF

(71) Applicant: SABIC Global Technologies B.V., Bergen op Zoom (NL)

(72) Inventors: Lynn COLUCCI-MIZENKO, Niskayuna, NY (US); Andrew KUGLER, Albany, NY (US)

(21) Appl. No.: 15/574,011

(22) PCT Filed: May 19, 2016
(86) PCT No.: PCT/US2016/033301

§ 371 Date: Nov. 14, 2017

Related U.S. Application Data
(60) Provisional application No. 62/163,966, filed on May 19, 2015.

(51) Int. Cl. A61L 31/06; A61L 31/16; A61L 31/02; A61N 1/375

(52) U.S. Cl. CPC A61L 31/06; A61L 31/16; A61L 31/024; A61L 2300/408; A61N 1/375; A61L 2300/404; A61L 2300/406; A61L 31/026

(57) ABSTRACT

Devices prepared from polyetherimide resins are disclosed. In one aspect, the article can be a medical device configured for use in a body.

### CROSS REFERENCE TO RELATED APPLICATION

This application claims the benefit of U.S. Provisional Application No. 62/163,966, filed May 19, 2015, the entirety of which is incorporated by reference herein.

### TECHNICAL FIELD

The disclosure concerns medical devices, prepared using thermoplastic resins, particularly resins comprising polyetherimide.

### BACKGROUND

For any type of medical device or article intended for contact with living tissue, the risk of foreign body reaction and of infection, among many other things, are paramount concerns. Considerations regarding the materials that make up the device can be crucial in order to avoid such adverse effects which can potentially lead to the failure of the device or harm the health of the receiving patient.

### SUMMARY

The application of thermoplastic resins in the medical field increasingly requires compositions able to the meet both the stringent physical requirements of typical polymer characteristics such as flow mechanical strength, as well biomedical considerations such as biocompatibility and stability. Accordingly, the materials selected to form the device are desired to exhibit certain characteristics, particularly biocompatibility and stability.

In an aspect, the present disclosure provides polyetherimide resins appropriate for use devices such as medical devices.

In another aspect, the disclosure concerns an article prepared according to the methods of forming a polyetherimide resin as disclosed herein.

In one aspect, the present disclosure provides polyetherimides for use in medical devices such as an implantable medical device.

In an aspect, the medical device can comprise a header, a body, leads, and one or more interior spacers.

### BRIEF DESCRIPTION OF THE FIGURES

FIG. 1 is a drawing of a medical device in accordance with an exemplary embodiment of present disclosure.

### DETAILED DESCRIPTION

Before the present methods and devices are disclosed and described, it is to be understood that the methods and devices are not limited to specific synthetic methods, specific components, or to particular compositions. It is also to be understood that the terminology used herein is for the purpose of describing particular embodiments only and is not intended to be limiting.

Medical devices as disclosed herein can include the use of polymer compositions and thermoplastic resins. The use of thermoplastic resins and polymer compositions in medical devices thus presents the balance of preserving structural integrity and ensuring biocompatibility with living tissue. The thermoplastic resins of the present disclosure can provide materials suitable for use in medical devices and articles.

### Polymer Composition

In one aspect of the disclosure, the medical device formed may be formed using a polymer composition. In one aspect of the present disclosure, the polymer composition can comprise a thermoplastic resin. Other components, however, may also be included in the thermoplastic resin. For example, the polymer composition may also include a ceramic and a metal.

In one aspect of the disclosure, the polymer composition can be suitable for melt processing such that the medical device, or a component thereof, can be formed using a melt process and in particular, injection molding. The polymer composition can include any polymeric material known in the art. The polymer composition may be composed of more than one polymeric material.

In one aspect of the disclosure, the polymers used in the polymer composition may be selected from a wide variety of thermoplastic polymers, and blends of thermoplastic polymers. The polymer composition can comprise a homopolymer, a copolymer such as a star block copolymer, a graft copolymer, an alternating block copolymer or a random copolymer, ionomer, dendrimer, or a combination comprising at least one of the foregoing. The polymer composition may also be a blend of polymers, copolymers, terpolymers, or the like, or a combination comprising at least one of the foregoing.

Examples of thermoplastic polymers that can be used in the polymer composition include polyacetals, polyacrylics, polycarbonates, polyalkyds, polystyrenes, polyolefins, polyesters, polyamides, polyaramides, polyamideimides, polyarylates, polyurethanes, epoxies, phenolics, silicones, polyarylsulfones, polyethersulfones, polyphenylene sulfides, polysulfones, polyimides, polyetherimides, polytetrafluoroethylenes, polyetherketones, polyether etherketones, polyether ketone ketones, polybenzoxazoles, polyoxadiazoles, polybenzothiazinophenothiazines, polybenzothiazoles, polypyrazinoquinoxalines, polypyromellitimides, polyquinoxalines, polybenzimidazoles, polyoxindoles, polyoxoisoindolines, polydioxoisoindolines, polytriazines, polypyridazines, polypiperazines, polypyridines, polypiperidines, polytriazoles, polypyrazoles, polycarboranes, polyoxabicyclononanes, polydibenzofurans, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, polypropylenes, polyethylenes, polyethylene terephthalates, polyvinylidene fluorides, polysiloxanes, or the like, or a combination comprising at least one of the foregoing thermoplastic polymers.

Examples of blends of thermoplastic polymers that can be used polymer composition resin include acrylonitrile-butadiene-styrene/nylon, polycarbonate/acrylonitrile-butadiene-styrene, polyphenylene ether/polystyrene, polyphenylene ether/polyamide, polycarbonate/polyester, polyphenylene ether/polyolefin, or the like, or a combination comprising at least one of the foregoing.

In one aspect of the present disclosure, polymer composition may include, polycarbonates, polysulfones, polyesters, polyamides, polypropylene. In a further aspect, the polyimides used in the disclosed polymer composition may include polyamideimides, polyetherimides and polybenzimidazoles. In a further aspect, polyetherimides comprise melt processable polyetherimides.

### Polyetherimide

In one aspect of the present disclosure, a medical device, or a component thereof, can be formed from a polyetherimide resin. Polyetherimides (“PEIs”) are amorphous, transparent, high performance polymers having a glass transition temperature (“Tg”) of greater than 180° C. PEIs further have high strength, heat resistance, and modulus, and broad chemical resistance. The high reliability and safety benefits afforded by a polyetherimide from its optical transparency, toughness, and heat resistance can be useful in medical applications.

In an aspect, polyetherimides can comprise polyetherimides homopolymers (e.g., polyetherimidesulfones) and polyetherimides copolymers. The polyetherimide can be selected from (i) polyetherimidehomopolymers, e.g., polyetherimides, (ii) polyetherimide co-polymers, and (iii) combinations thereof. Polyetherimides are known polymers and are sold by SABIC Innovative Plastics under the ULTEM®*, EXTEM®*, and Siltem* brands (Trademark of SABIC Innovative Plastics IP B.V.).

In an aspect, the polyetherimides can be of formula (1):

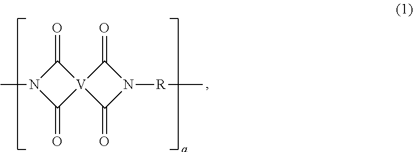

wherein a is more than 1, for example 10 to 1,000 or more, or more specifically 10 to 500.

The group V in formula (1) is a tetravalent linker containing an ether group (a “polyetherimide” as used herein) or a combination of an ether groups and arylenesulfone groups (a “polyetherimidesulfone”). Such linkers include but are not limited to: (a) substituted or unsubstituted, saturated, unsaturated or aromatic monocyclic and polycyclic groups having 5 to 50 carbon atoms, optionally substituted with ether groups, arylenesulfone groups, or a combination of ether groups and arylenesulfone groups; and (b) substituted or unsubstituted, linear or branched, saturated or unsaturated alkyl groups having 1 to 30 carbon atoms and optionally substituted with ether groups or a combination of ether groups, arylenesulfone groups, and arylenesulfone groups; or combinations comprising at least one of the foregoing. Suitable additional substitutions include, but are not limited to, ethers, amides, esters, and combinations comprising at least one of the foregoing.

The R group in formula (1) includes but is not limited to substituted or unsubstituted divalent organic groups such as: (a) aromatic hydrocarbon groups having 6 to 20 carbon atoms and halogenated derivatives thereof; (b) straight or branched chain alkylene groups having 2 to 20 carbon atoms; (c) cycloalkylene groups having 3 to 20 carbon atoms, or (d) divalent groups of formula (2):

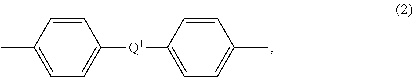

wherein Q1 includes but is not limited to a divalent moiety such as —O—, —S—, —C(O)—, —SO2-, —SO—, —CyH2y- (y being an integer from 1 to 5), and halogenated derivatives thereof, including perfluoroalkylene groups.

In an embodiment, linkers V include but are not limited to tetravalent aromatic groups of formula (3):

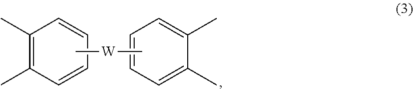

wherein W is a divalent moiety including —O—, —SO2-, or a group of the formula —O—Z—O— wherein the divalent bonds of the —O— or the —O—Z—O— group are in the 3,3′, 3,4′, 4,3′, or the 4,4′ positions, and wherein Z includes, but is not limited, to divalent groups of formulas (4):

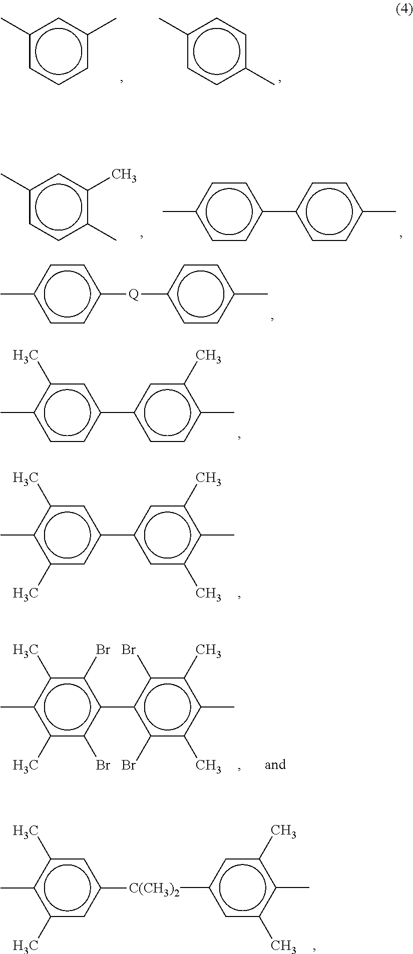

wherein Q includes, but is not limited to a divalent moiety including —O—, —S—, —C(O), —SO₂—, —SO—, —C_{y}H_{2y}— (y being an integer from 1 to 5), and halogenated derivatives thereof, including perfluoroalkylene groups.

In an aspect, the polyetherimide comprise more than 1, specifically 10 to 1,000, or more specifically, 10 to 500 structural units, of formula (5):

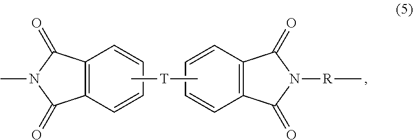

wherein T is —O— or a group of the formula —O—Z—O— wherein the divalent bonds of the —O— or the —O—Z—O— group are in the 3,3′, 3,4′, 4,3′, or the 4,4′ positions; Z is a divalent group of formula (3) as defined above; and R is a divalent group of formula (2) as defined above.

In another aspect, the polyetherimidesulfones are polyetherimides comprising ether groups and sulfone groups wherein at least 50 mole % of the linkers V and the groups R in formula (1) comprise a divalent arylenesulfone group. For example, all linkers V, but no groups R, can contain an arylenesulfone group; or all groups R but no linkers V can contain an arylenesulfone group; or an arylenesulfone can be present in some fraction of the linkers V and R groups, provided that the total mole fraction of V and R groups containing an aryl sulfone group is greater than or equal to 50 mole %.

Even more specifically, polyetherimidesulfones can comprise more than 1, specifically 10 to 1,000, or more specifically, 10 to 500 structural units of formula (6):

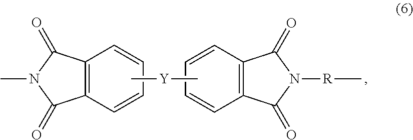

wherein Y is —O—, —SO2-, or a group of the formula —O—Z—O— wherein the divalent bonds of the —O—, SO2-, or the —O—Z—O— group are in the 3,3′, 3,4′, 4,3′, or the 4,4′ positions, wherein Z is a divalent group of formula (3) as defined above and R is a divalent group of formula (2) as defined above, provided that greater than 50 mole % of the sum of moles Y+moles R in formula (2) contain —SO2- groups.

It is to be understood that the polyetherimides and polyetherimidesulfones can optionally comprise linkers V that do not contain ether or ether and sulfone groups, for example linkers of formula (7):

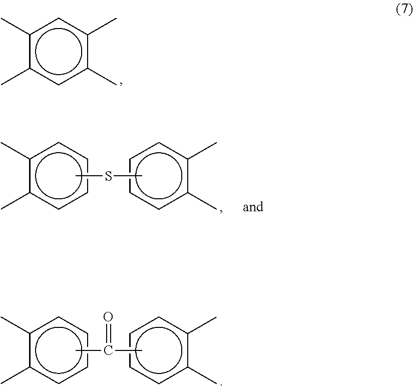

Imide units containing such linkers are generally be present in amounts ranging from 0 to 10 mole % of the total number of units, specifically 0 to 5 mole %. In one embodiment no additional linkers V are present in the polyetherimides.

In another aspect, the polyetherimide comprises 10 to 500 structural units of formula (5) and the polyetherimidesulfone contains 10 to 500 structural units of formula (6).

Polyetherimides can be prepared by any suitable process. In one embodiment, polyetherimides and polyetherimide copolymers include polycondensation polymerization processes and halo-displacement polymerization processes.

Polycondensation methods can include a method for the preparation of polyetherimides having structure (1) is referred to as the nitro-displacement process (X is nitro in formula (8)). In one example of the nitro-displacement process, N-methyl phthalimide is nitrated with 99% nitric acid to yield a mixture of N-methyl-4-nitrophthalimide (4-NPI) and N-methyl-3-nitrophthalimide (3-NPI). After purification, the mixture, containing approximately 95 parts of 4-NPI and 5 parts of 3-NPI, is reacted in toluene with the disodium salt of bisphenol-A (BPA) in the presence of a phase transfer catalyst. This reaction yields BPA-bisimide and NaNO₂ in what is known as the nitro-displacement step. After purification, the BPA-bisimide is reacted with phthalic anhydride in an imide exchange reaction to afford BPA-dianhydride (BPADA), which in turn is reacted with a diamine such as meta-phenylene diamine (MPD) in ortho-dichlorobenzene in an imidization-polymerization step to afford the product polyetherimide.

Other diamines are also possible. Examples of suitable diamines include: m-phenylenediamine; p-phenylenediamine; 2,4-diaminotoluene; 2,6-diaminotoluene; m-xylylenediamine; p-xylylenediamine; benzidine; 3,3′-dimethylbenzidine; 3,3′-dimethoxybenzidine; 1,5-diaminonaphthalene; bis(4-aminophenyl)methane; bis(4-aminophenyl)propane; bis(4-aminophenyl)sulfide; bis(4-aminophenyl)sulfone; bis(4-aminophenyl)ether; 4,4′-diaminodiphenylpropane; 4,4′-diaminodiphenylmethane(4,4′-methylenedianiline); 4,4′-diaminodiphenyl sulfide; 4,4′-diaminodiphenylsulfone; 4,4′-diaminodiphenylether(4,4′-oxydianiline); 1,5-diaminonaphthalene; 3,3′dimethylbenzidine; 3-methylheptamethylenediamine; 4,4-dimethylheptamethylenediamine; 2,2′,3,3′-tetrahydro-3,3,3′,3′-tetramethyl-1,1′-spirobi[1H-indene]-6,6′-diamine; 3,3′,4,4′-tetrahydro-4,4,4′,4′-tetramethyl-2,2′-spirobi[2H-1-benzo-pyran]-7,7′-diamine; 1,1′-bis[1-amino-2-methyl-4-phenyl]cyclohexane, and isomers thereof as well as mixtures and blends comprising at least one of the foregoing. In one embodiment, the diamines are specifically aromatic diamines, especially m- and p-phenylenediamine and mixtures comprising at least one of the foregoing.

Suitable dianhydrides that can be used with the diamines include and are not limited to 2,2-bis[4-(3,4-dicarboxyphenoxy)phenyl]propane dianhydride; 4,4′-bis(3,4-dicarboxyphenoxy)diphenyletherdianhydride; 4,4′-bis(3,4-dicarboxyphenoxy)diphenylsulfiondedianhydride; 4,4′-bis(3,4-dicarboxyphenoxy)benzophenonedianhydride; 4,4′-bis(3,4-dicarboxyphenoxy)diphenyl sulfonedianhydride; 2,2-bis[4-(2,3-dicarboxyphenoxy)phenyl]propane dianhydride; 4,4′-bis(2,3-dicarboxyphenoxy)diphenyletherdianhydride; 4,4′-bis(2,3-dicarboxyphenoxy)diphenylsulfidedianhydride; 4,4′-bis(2,3-dicarboxyphenoxy)benzophenonedianhydride; 4,4′-bis(2,3-dicarboxyphenoxy)diphenylsulfonedianhydride; 4-(2,3-dicarboxyphenoxy)-4′-(3,4-dicarboxyphenoxy)diphenyl-2,2-propane dianhydride; 4-(2,3-dicarboxyphenoxy)-4′-(3,4-dicarboxyphenoxy)diphenyletherdianhydride; 4-(2,3-dicarboxyphenoxy)-4′-(3,4-dicarboxyphenoxy)diphenylsulfide dianhydride; 4-(2,3-dicarboxyphenoxy)-4′-(3,4-dicarboxyphenoxy)benzophenonedianhydride; 4-(2,3-dicarboxyphenoxy)-4′-(3,4-dicarboxyphenoxy)diphenylsulfone dianhydride; 1,3-bis(2,3-dicarboxyphenoxy)benzene dianhydride; 1,4-bis(2,3-dicarboxyphenoxy)benzene dianhydride; 1,3-bis(3,4-dicarboxyphenoxy)benzene dianhydride; 1,4-bis(3,4-dicarboxyphenoxy)benzene dianhydride; 3,3′,4,4′-diphenyl tetracarboxylicdianhydride; 3,3′,4,4′-benzophenonetetracarboxylic dianhydride; naphthalicdianhydrides, such as 2,3,6,7-naphthalic dianhydride, etc.; 3,3′,4,4′-biphenylsulphonictetracarboxylic dianhydride; 3,3′,4,4′-biphenylethertetracarboxylic dianhydride; 3,3′,4,4′-dimethyldiphenylsilanetetracarboxylic dianhydride; 4,4′-bis (3,4-dicarboxyphenoxy)diphenylsulfidedianhydride; 4,4′-bis (3,4-dicarboxyphenoxy)diphenyl sulphonedianhydride; 4,4′-bis (3,4-dicarboxyphenoxy)diphenylpropanedianhydride; 3,3′,4,4′-biphenyltetracarboxylic dianhydride; bis(phthalic)phenylsulphineoxidedianhydride; p-phenylene-bis(triphenylphthalic)dianhydride; m-phenylene-bis(triphenylphthalic)dianhydride; bis(triphenylphthalic)-4,4′-diphenylether dianhydride; bis(triphenylphthalic)-4,4′-diphenylmethane dianhydride; 2,2′-bis(3,4-dicarboxyphenyl)hexafluoropropanedianhydride; 4,4′-oxydiphthalic dianhydride; pyromelliticdianhydride; 3,3′,4,4′-diphenylsulfonetetracarboxylic dianhydride; 4′,4′-bisphenol A dianhydride; hydroquinone diphthalic dianhydride; 6,6′-bis(3,4-dicarboxyphenoxy)-2,2′,3,3′-tetrahydro-3,3,3′,3′-tetramethyl-1,1′-spirobi[1H-indene]dianhydride; 7,7′-bis(3,4-dicarboxyphenoxy)-3,3′,4,4′-tetrahydro-4,4,4′,4′-tetramethyl-2,2′-spirobi[2H-1-benzopyran]dianhydride; 1,1′-bis[1-(3,4-dicarboxyphenoxy)-2-methyl-4-phenyl]cyclohexane dianhydride; 3,3′,4,4′-diphenylsulfonetetracarboxylic dianhydride; 3,3′,4,4′-diphenylsulfidetetracarboxylic dianhydride; 3,3′,4,4′-diphenylsulfoxidetetracarboxylic dianhydride; 4,4′-oxydiphthalic dianhydride; 3,4′-oxydiphthalic dianhydride; 3,3′-oxydiphthalic dianhydride; 3,3′-benzophenonetetracarboxylic dianhydride; 4,4′-carbonyldiphthalic dianhydride; 3,3′,4,4′-diphenylmethanetetracarboxylic dianhydride; 2,2-bis(4-(3,3-dicarboxyphenyl)propane dianhydride; 2,2-bis(4-(3,3-dicarboxyphenyl)hexafluoropropanedianhydride; (3,3′,4,4′-diphenyl)phenylphosphinetetracarboxylicdianhydride; (3,3′,4,4′-diphenyl)phenylphosphineoxidetetracarboxylicdianhydride; 2,2′-dichloro-3,3′,4,4′-biphenyltetracarboxylic dianhydride; 2,2′-dimethyl-3,3′,4,4′-biphenyltetracarboxylic dianhydride; 2,2′-dicyano-3,3′,4,4′-biphenyltetracarboxylic dianhydride; 2,2′-dibromo-3,3′,4,4′-biphenyltetracarboxylic dianhydride; 2,2′-diiodo-3,3′,4,4′-biphenyltetracarboxylic dianhydride; 2,2′-ditrifluoromethyl-3,3′,4,4′-biphenyltetracarboxylic dianhydride; 2,2′-bis(1-methyl-4-phenyl)-3,3′,4,4′-biphenyltetracarboxylic dianhydride; 2,2′-bis(1-trifluoromethyl-2-phenyl)-3,3′,4,4′-biphenyltetracarboxylic dianhydride; 2,2′-bis(1-trifluoromethyl-3-phenyl)-3,3′,4,4′-biphenyltetracarboxylic dianhydride; 2,2′-bis(1-trifluoromethyl-4-phenyl)-3,3′,4,4′-biphenyltetracarboxylic dianhydride; 2,2′-bis(1-phenyl-4-phenyl)-3,3′,4,4′-biphenyltetracarboxylic dianhydride; 4,4′-bisphenol A dianhydride; 3,4′-bisphenol A dianhydride; 3,3′-bisphenol A dianhydride; 3,3′,4,4′-diphenylsulfoxidetetracarboxylic dianhydride; 4,4′-carbonyldiphthalic dianhydride; 3,3′,4,4′-diphenylmethanetetracarboxylic dianhydride; 2,2′-bis(1,3-trifluoromethyl-4-phenyl)-3,3′,4,4′-biphenyltetracarboxylic dianhydride, and all isomers thereof, as well as combinations of the foregoing.

Halo-displacement polymerization methods for making polyetherimides and polyetherimidesulfones include and are not limited to, the reaction of a bis(phthalimide) for formula (8):

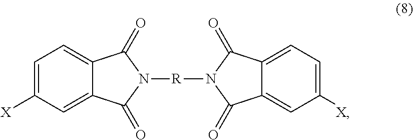

wherein R is as described above and X is a nitro group or a halogen. Bis-phthalimides (8) can be formed, for example, by the condensation of the corresponding anhydride of formula (9):

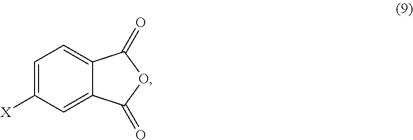

wherein X is a nitro group or halogen, with an organic diamine of the formula (10):

H₂N—R—NH₂ (10),

wherein R is as described above.

Illustrative examples of amine compounds of formula (10) include: ethylenediamine, propylenediamine, trimethylenediamine, diethylenetriamine, triethylenetetramine, hexamethylenediamine, heptamethylenediamine, octamethylenediamine, nonamethylenediamine, decamethylenediamine, 1,12-dodecanediamine, 1,18-octadecanediamine, 3-methylheptamethylenediamine, 4,4-dimethylheptamethylenediamine, 4-methylnonamethylenediamine, 5-methylnonamethylenediamine, 2,5-dimethylhexamethylenediamine, 2,5-dimethylheptamethylenediamine, 2, 2-dimethylpropylenediamine, N-methyl-bis (3-aminopropyl) amine, 3-methoxyhexamethylenediamine, 1,2-bis(3-aminopropoxy) ethane, bis(3-aminopropyl) sulfide, 1,4-cyclohexanediamine, bis-(4-aminocyclohexyl) methane, m-phenylenediamine, p-phenylenediamine, 2,4-diaminotoluene, 2,6-diaminotoluene, m-xylylenediamine, p-xylylenediamine, 2-methyl-4,6-diethyl-1,3-phenylene-diamine, 5-methyl-4,6-diethyl-1,3-phenylene-diamine, benzidine, 3,3′-dimethylbenzidine, 3,3′-dimethoxybenzidine, 1,5-diaminonaphthalene, bis(4-aminophenyl) methane, bis(2-chloro-4-amino-3, 5-diethylphenyl) methane, bis(4-aminophenyl) propane, 2,4-bis(b-amino-t-butyl) toluene, bis(p-b-amino-t-butylphenyl) ether, bis(p-b-methyl-o-aminophenyl) benzene, bis(p-b-methyl-o-aminopentyl) benzene, 1, 3-diamino-4-isopropylbenzene, bis(4-aminophenyl) ether and 1,3-bis(3-aminopropyl) tetramethyldisiloxane. Mixtures of these amines can be used. Illustrative examples of amine compounds of formula (10) containing sulfone groups include but are not limited to, diaminodiphenylsulfone (DDS) and bis(aminophenoxy phenyl) sulfones (BAPS). Combinations comprising any of the foregoing amines can be used.

The polyetherimides can be synthesized by the reaction of the bis(phthalimide) (8) with an alkali metal salt of a dihydroxy substituted aromatic hydrocarbon of the formula HO—V—OH wherein V is as described above, in the presence or absence of phase transfer catalyst. Suitable phase transfer catalysts are disclosed in U.S. Pat. No. 5,229,482. Specifically, the dihydroxy substituted aromatic hydrocarbon a bisphenol such as bisphenol A, or a combination of an alkali metal salt of a bisphenol and an alkali metal salt of another dihydroxy substituted aromatic hydrocarbon can be used.

In one embodiment, the polyetherimide comprises structural units of formula (5) wherein each R is independently p-phenylene or m-phenylene or a mixture comprising at least one of the foregoing; and T is group of the formula —O—Z—O— wherein the divalent bonds of the —O—Z—O— group are in the 3,3′ positions, and Z is 2,2-diphenylenepropane group (a bisphenol A group). Further, the polyetherimidesulfone comprises structural units of formula (6) wherein at least 50 mole % of the R groups are of formula (4) wherein Q is —SO2- and the remaining R groups are independently p-phenylene or m-phenylene or a combination comprising at least one of the foregoing; and T is group of the formula —O—Z—O— wherein the divalent bonds of the —O—Z—O— group are in the 3,3′ positions, and Z is a 2,2-diphenylenepropane group.

The polyetherimide and polyetherimidesulfone can be used alone or in combination with each other and/or other of the disclosed polymeric materials in fabricating the polymeric components of the invention. In one embodiment, only the polyetherimide is used. In another embodiment, the weight ratio of polyetherimide:polyetherimidesulfone can be from 99:1 to 50:50.

The polyetherimides can have a weight average molecular weight (Mw) of 5,000 to 100,000 grams per mole (g/mole) as measured by gel permeation chromatography (GPC). In some embodiments the Mw can be 10,000 to 80,000. The molecular weights as used herein refer to the absolute weight averaged molecular weight (Mw).

The polyetherimides can have an intrinsic viscosity greater than or equal to 0.2 deciliters per gram (dl/g) as measured in m-cresol at 25° C. Within this range the intrinsic viscosity can be 0.35 to 1.0 dl/g, as measured in m-cresol at 25° C.

The polyetherimides can have a glass transition temperature of greater than 180° C., specifically of 200° C. to 500° C., as measured using differential scanning calorimetry (DSC) per ASTM test D3418. In some embodiments, the polyetherimide and, in particular, a polyetherimide has a glass transition temperature of 240 to 350° C.

The polyetherimides can have a melt index of 0.1 to 10 grams per minute (g/min), as measured by American Society for Testing Materials (ASTM) DI 238 at 340 to 370° C., using a 6.7 kilogram (kg) weight.

In certain aspects, the polyetherimides of the present disclosure may be unfilled, standard flow grades (PEI-1 in Tables 1-2) or unfilled, high flow grades (PEI-2 in Tables 1-2), or may be filled, for example, with carbon (e.g., carbon fiber) or glass. Filled polymer components may include between 40 wt % and 90 wt % of the polyetherimide resin and between 10 wt % and 60 wt % of a filler by weight of the polymer component. Other formulations may be used.

An alternative halo-displacement polymerization process for making polyetherimides, e.g., polyetherimides having structure (1) is a process referred to as the chloro-displacement process (X is Cl in formula (8)). The chloro-displacement process is illustrated as follows: 4-chloro phthalic anhydride and meta-phenylene diamine are reacted in the presence of a catalytic amount of sodium phenyl phosphinate catalyst to produce the bischlorophthalimide of meta-phenylene diamine (CAS No. 148935-94-8). The bischlorophthalimide is then subjected to polymerization by chloro-displacement reaction with the disodium salt of BPA in the presence of a catalyst in ortho-dichlorobenzene or anisole solvent. Alternatively, mixtures of 3-chloro- and 4-chlorophthalic anhydride may be employed to provide a mixture of isomeric bischlorophthalimides which may be polymerized by chloro-displacement with BPA disodium salt as described above.

Siloxane polyetherimides can include polysiloxane/polyetherimide block or random copolymers having a siloxane content of greater than 0 and less than 40 weight percent (wt %) based on the total weight of the block copolymer. The block copolymer comprises a siloxane block of Formula (I):

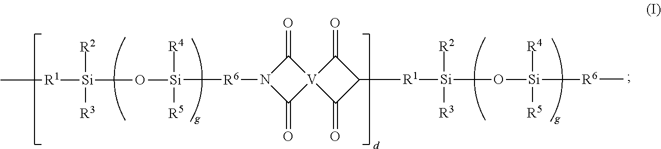

wherein R¹⁻⁶ are independently at each occurrence selected from the group consisting of substituted or unsubstituted, saturated, unsaturated, or aromatic monocyclic groups having 5 to 30 carbon atoms, substituted or unsubstituted, saturated, unsaturated, or aromatic polycyclic groups having 5 to 30 carbon atoms, substituted or unsubstituted alkyl groups having 1 to 30 carbon atoms and substituted or unsubstitutedalkenyl groups having 2 to 30 carbon atoms, V is a tetravalent linker selected from the group consisting of substituted or unsubstituted, saturated, unsaturated, or aromatic monocyclic and polycyclic groups having 5 to 50 carbon atoms, substituted or unsubstituted alkyl groups having 1 to 30 carbon atoms, substituted or unsubstitutedalkenyl groups having 2 to 30 carbon atoms and combinations comprising at least one of the foregoing linkers, g equals 1 to 30, and d is 2 to 20. Commercially available siloxane polyetherimides can be obtained from SABIC Innovative Plastics under the brand name SILTEM* (*Trademark of SABIC Innovative Plastics IP B.V.)

The polyetherimide resin can have a weight average molecular weight (Mw) within a range having a lower limit and/or an upper limit. The range can include or exclude the lower limit and/or the upper limit. The lower limit and/or upper limit can be selected from 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 101000, 102000, 103000, 104000, 105000, 106000, 107000, 108000, 109000, and 110000 daltons. For example, the polyetherimide resin can have a weight average molecular weight (Mw) from 5,000 to 100,000 daltons, from 5,000 to 80,000 daltons, or from 5,000 to 70,000 daltons. The primary alkyl amine modified polyetherimide will have lower molecular weight and higher melt flow than the starting, unmodified, polyetherimide.

The polyetherimide resin can be selected from the group consisting of a polyetherimide, for example as described in U.S. Pat. Nos. 3,875,116; 6,919,422 and 6,355,723 a silicone polyetherimide, for example as described in U.S. Pat. Nos. 4,690,997; 4,808,686 a polyetherimidesulfone resin, as described in U.S. Pat. No. 7,041,773 and combinations thereof, each of these patents are incorporated herein their entirety.

The polyetherimide resin can have a glass transition temperature within a range having a lower limit and/or an upper limit. The range can include or exclude the lower limit and/or the upper limit. The lower limit and/or upper limit can be selected from 100, 110, 120, 130, 140, 150, 160, 170, 180, 190, 200, 210, 220, 230, 240, 250, 260, 270, 280, 290, 300 and 310 degrees Celsius. For example, the polyetherimide resin can have a glass transition temperature (Tg) greater than about 200 degrees Celsius.

The polyetherimide resin can be substantially free (less than 100 ppm) of benzylic protons. The polyetherimide resin can be free of benzylic protons. The polyetherimide resin can have an amount of benzylic protons below 100 ppm. In one embodiment, the amount of benzylic protons ranges from more than 0 to below 100 ppm. In another embodiment, the amount of benzylic protons is not detectable.

The polyetherimide resin can be substantially free (less than 100 ppm) of halogen atoms. The polyetherimide resin can be free of halogen atoms. The polyetherimide resin can have an amount of halogen atoms below 100 ppm. In one embodiment, the amount of halogen atoms range from more than 0 to below 100 ppm. In another embodiment, the amount of halogen atoms is not detectable.

In various aspects, the polyetherimide polymer can be used in a number of forms. As an example, the polyetherimide resin can comprise continuous polymeric filaments or fibers. In further examples, the polyetherimide resin can comprise sheets.

### Additional Components

In an aspect, the thermoplastic resin used to form the medical device, or a part thereof, can include certain additional components. In one aspect, the thermoplastic resin can include a biocide.

### Properties and Articles

The present disclosure relates to articles comprising thermoplastic resins disclosed herein. In certain aspects, the articles can be formed from polyetherimide resins as described herein. The advantageous mechanical characteristics of the polyetherimides disclosed herein can make them appropriate for an array of articles. Suitable articles can be exemplified by, but are not limited to automotive components, including exterior and interior components; domestic appliances; enclosures for electrical and telecommunication devices; marine equipment; and medical instruments. The disclosure further contemplates additional fabrication operations on said articles.

In one aspect of the present disclosure, the article can comprise a medical device, such as an implantable medical device, or a component thereof, prepared from polyetherimide. As an example, the medical device can be used to provide diagnostics or to deliver treatment and can include one or more electrodes coupled to circuitry located on or within the device. The circuitry can be configured to monitor the electrical activity of a given organ to which it is connected. Exemplary implantable medical devices comprising polyetherimide can include cardiac rhythm management devices such as implantable pacemakers, implantable defibrillators (such as, implantable cardioverter-defibrillators (ICDSs)), cardiac resynchronization therapy devices (CRTSs), neural stimulators and modulators, or one or more other devices.

In an exemplary aspect, the implantable medical device comprising a thermoplastic resin be configured to diagnose and modify cardiac function. Electrical stimulus in a normally operating heart drives the blood pumping function of the organ. The electrical stimulus is generated within the right atrium and transmitted to the ventricle where the stimulus provides a contracting or beating of the ventricle. Aging and cardiovascular disease, among other conditions, can obstruct the electrical stimulus in the heart and prevent the heart from beating at its normal rate oftentimes resulting in fatigue, severe illness, or death. As an example, an implantable medical device can be placed within the cardiac tissue to monitor the cardiac activity and to detect when electrical stimulus has been obstructed. When obstruction becomes apparent, the device can provide electrical stimulus to the heart until the organ regains the ability to effectively operate. As such, the device can utilize a powersource and circuitry to deliver the electrical therapy to elicit the necessary stimuli.

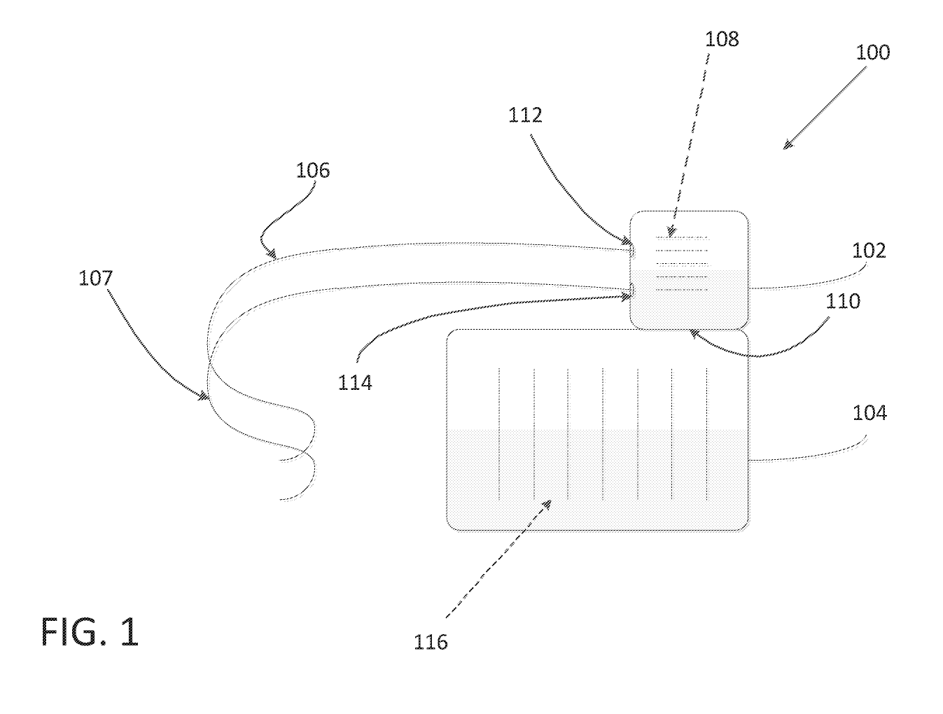

In various aspects, the medical device can comprise a header, a body or enclosure, and one or more leads. FIG. 1 provides an exemplary implantable medical device 100. The medical device 100 may comprise a header 102, an enclosure 104, and one or more leads 106. The header 102 of the medical device can be coupled to the enclosure 104. The header 102 can further comprise one or more electrical contacts 108. The electrical contacts 108 can in turn be connected to one or more leads 106. The header can include one or more bores 112, 114 or openings to allow passage of the leads 106 through the header 102.

In an aspect, the header 102 can be coupled to the enclosure 104 to facilitate electrical communication between elements within the sealed enclosure 104 and elements external to the sealed enclosure 104. The coupling of the header 102 and the enclosure 104 can allow passage of the leads 106 from the electrical contacts 108 into the enclosure 104 to connect to the power source and circuitry situated therein.

The header 102 can be formed from a resin that is molded and cured into desired configuration. In an exemplary method, the header 102 can be molded separately from the enclosure 104. In other methods, the header 102 can be molded while in contact with the enclosure. In one example, the header 102 can be transparent. A transparent header 102 can be useful because components such as the electrical contacts 108 can be visibly inspected.

In an aspect, the enclosure 104 can be configured to house circuitry and a power source necessary to monitor and modify cardiac function or other biological functions. The powersource can comprise one or more electrochemical cells and operative electric circuitry within the enclosure 104. The enclosure of the implantable medical device can be hermetically sealed to protect the powersource and circuitry components from corrosive bodily fluids.

In various aspects, a spacer member or one or more spacers 116 can be disposed within the enclosure to situate the powersource comprising one or more electrochemical cells and operative electric circuitry. The spacers 116 can be formed from a thermoplastic resin. In one example, the one or more spacers can be formed from a polyetherimide resin. This spacers 116 may be generally planar and may be provided to electrically isolate components within the enclosure 104 (e.g., in the can), absorb tolerances, and operate as a shock absorber. Other components internal to the enclosure 104 may be fored from a polyetherimide resin for similar benefits as the spacers 116.

The leads 106 can be configured to direct an electrical stimulus from enclosure 104 to the tissue to which they are connected. In an aspect, the leads 106 can be connected to the circuitry in the enclosure 104. Via the connection to the circuitry, the leads 106 can deliver an electrical impulse generated by the powersource to connected tissue. In a further example, the leads 104 can be attached to cardiac tissue to monitor and to generate electrical activity.

In one aspect, the medical device or parts thereof can be formed from a thermoplastic resin. More specifically, the medical device or parts thereof can be formed from polyetherimide resin. In an example, the header 102 can be formed from a polyetherimide. In a further example, the spacers can be formed from a polyetherimide resin.

The patentable scope of the invention is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

As an illustrative example, the polyetherimides used in forming the apparatus of the present disclosure may exhibit distinguishable properties over other comparative polymers, as shown in Tables 1-2 (PEI—polyetherimide; PPSU—polyphenylsulfone; PSU—polysulfone; PEEK—Polyether ether ketone; TPU—thermoplastic polyurethane):

TABLE 1

| | | | E1 | E2 | CE1 | CE2 | CE3 |
|---|---|---|---|---|---|---|---|
| | | | Polymer Type | | | | |
| MECHANICAL | Unit | Standard | PEI-1 | PEI-2 | PPSU | PSU | PEEK |
| Tensile Stress @ | kgf/cm² | ASTM D | 1120 | 1120 | 710 | 720 | 1020 |
| Yield, Type I, 5 | | 638 | | | | | |
| mm/min | | | | | | | |
| Tensile Modulus, | kgf/cm² | ASTM D | 36500 | 36500 | 23900 | 25300 | 37700 |
| 5 mm/min | | 638 | | | | | |
| Flexural Stress | kgf/cm² | ASTM D | 1760 | 1770 | 930 | 1080 | 1560 |
| @ Yield, 1.3 | | 790 | | | | | |
| mm/min, 50 mm | | | | | | | |
| span | | | | | | | |
| Flexural | kgf/cm² | ASTM D | 35000 | 34900 | 24600 | 27400 | 38700 |
| Modulus, 1.3 | | 790 | | | | | |
| mm/min, 50 mm | | | | | | | |
| span | | | | | | | |

| IMPACT | Unit | Standard | Value | | | | |
|---|---|---|---|---|---|---|---|
| Izod Impact, | cm- | ASTM D | 5 | 3 | 70 | 7.0 | 5.4 |
| notched, 23° C. | kgf/cm | 256 | | | | | |

| PHYSICAL | Unit | Standard | | Value | | | |
|---|---|---|---|---|---|---|---|
| Specific Gravity | — | ASTM D | 1.27 | 1.27 | 1.29 | 1.24 | 1.30 |
| | | 792 | | | | | |
| Melt Flow Rate, | g/10 min | ASTM D | — | — | — | — | 36 |
| 400° C./2.16 kgf | | 1238 | | | | | |
| Melt Flow Rate, | g/10 min | ASTM D | — | — | 14-20 | — | — |
| 365° C./5.0 kgf | | 1238 | | | | | |
| Melt Flow Rate, | g/10 min | ASTM D | — | — | — | 6.5 | — |
| 343° C./2.16 kgf | | 1238 | | | | | |
| Melt Flow Rate, | g/10 min | ASTM D | 9 | 17.8 | — | — | — |
| 337° C./6.6 kgf | | 1238 | | | | | |

| ELECTRICAL | Unit | Standard | | Value | | | |
|---|---|---|---|---|---|---|---|
| Volume | Ohm- | ASTM D | 1.00E+17 | 1.00E+17 | 9.00E+15 | 3.00E+16 | — |
| Resistivity | cm | 257 | | | | | |

| THERMAL | Unit | Standard | | Value | | | |
|---|---|---|---|---|---|---|---|
| Glass Transition | ° C. | | 217 | 217 | 220 | — | 147 |
| Temperature | | | | | | | |
| Heat Deflection | ° C. | ASTM D | 201 | 198 | 207 | 174 | 160 |
| Temperature, | | 648 | | | | | |
| 1.82 MPa | | | | | | | |

TABLE 2

| | | | E1 | E2 | CE4 | CE5 | CE6 |
|---|---|---|---|---|---|---|---|
| | | | Polymer Type | | | | |
| MECHANICAL | Unit | Standard | PEI-1 | PEI-2 | TPU | TPU | TPU |
| Tensile Stress @ | kgf/cm² | ASTM D | — | — | — | 720 | 1020 |
| Yield, Type I, 5 | | 638 | | | | | |
| mm/min | | | | | | | |
| Tensile Modulus, | kgf/cm² | ASTM D | — | — | — | 25300 | 37700 |
| 5 mm/min | | 638 | | | | | |
| Flexural Stress | kgf/cm² | ASTM D | 16 | 63 | 770 | 1080 | 1560 |
| @ Yield, 1.3 | | 790 | | | | | |
| mm/min, 50 mm | | | | | | | |
| span | | | | | | | |
| Flexural | kgf/cm² | ASTM D | 370 | 1520 | 20320 | 27400 | 38700 |
| Modulus, 1.3 | | 790 | | | | | |
| mm/min, 50 mm | | | | | | | |
| span | | | | | | | |

| IMPACT | Unit | Standard | | | | | |
|---|---|---|---|---|---|---|---|
| Izod Impact, | cm- | ASTM D | — | — | — | 7.0 | 5.4 |
| notched, 23° C. | kgf/cm | 256 | | | | | |

| PHYSICAL | Unit | Standard | | | | | |
|---|---|---|---|---|---|---|---|
| Specific Gravity | — | ASTM D | 1.12 | 1.16 | 1.19 | 1.24 | 1.30 |
| | | 792 | | | | | |
| Melt Flow Rate, | g/10 min | ASTM D | — | — | — | — | — |
| 400° C./2.16 kgf | | 1238 | | | | | |
| Melt Flow Rate, | g/10 min | ASTM D | — | — | — | — | — |
| 365° C./5.0 kgf | | 1238 | | | | | |
| Melt Flow Rate, | g/10 min | ASTM D | — | — | — | — | — |
| 343° C./2.16 kgf | | 1238 | | | | | |
| Melt Flow Rate, | g/10 min | ASTM D | 9 | 17.8 | — | — | — |
| 337° C./6.6 kgf | | 1238 | | | | | |
| Melt Flow Rate, | g/10 min | ASTM D | — | — | 17 | 13 | 37 |
| 224° C. | | 1238 | | | | | |

| ELECTRICAL | Unit | Standard | | | | | |
|---|---|---|---|---|---|---|---|
| Volume | Ohm- | ASTM D | — | — | — | 3.00E+16 | — |
| Resistivity | cm | 257 | | | | | |

| THERMAL | Unit | Standard | | | | | |
|---|---|---|---|---|---|---|---|
| Glass Transition | ° C. | | — | — | — | — | 147 |
| Temperature | | | | | | | |
| Heat Deflection | ° C. | ASTM D | — | — | — | 174 | 160 |
| Temperature, | | 648 | | | | | |
| 1.82 MPa | | | | | | | |

### Methods

In certain aspects of the disclosure, the medical device or a part thereof can be formed by any method or combination of methods known in the art. These methods include, but are not limited to, molding processes, additive manufacturing, and machining. These molding processes include, but are not limited to, various melt forming process, injection molding, blow molding (stretch, extrusion or injection), sheet and film extrusion, profile extrusion, thermoforming, additive manufacturing, compression molding, fiber extrusion, powder sintering, transfer molding, reaction injection (RIM) molding, vacuum forming, cold casting, dip molding, slush molding and press molding. In one aspect, a combination of these molding methods may be used to form the header or the one or more spacers.

In various aspects, a thermoplastic resin as disclosed herein can be prepared according to a variety of methods for use in the medical device. As an example, a polyetherimide resin can be molded to form the header or the spacer by a variety of means such as injection molding, extrusion, rotational molding, blow molding and thermoforming to form the header. The polyetherimide resins of the present disclosure can be blended, compounded, or otherwise combined by a variety of methods involving intimate admixing of the materials with any additional additives desired in the formulation. Because of the availability of melt blending equipment in commercial polymer processing facilities, melt processing methods can be used. In various further aspects, the equipment used in such melt processing methods can include, but is not limited to, the following: co-rotating and counter-rotating extruders, single screw extruders, co-kneaders, disc-pack processors and various other types of extrusion equipment. In a further aspect, the extruder is a twin-screw extruder. In various further aspects, the thermoplastic composition can be processed in an extruder at temperatures from about 180° C. to about 350° C.

While aspects of the present disclosure can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

### Aspects

The present disclosure comprises at least the following aspects.

Aspect 1: A medical device comprising: a header comprising one or more bores; one or more leads disposed in the header and exiting the header through the one or more bores; an enclosure coupled to the header, the enclosure comprising circuitry, a power source, and one or more spacers, wherein the one or more spacers are formed from a polyetherimide resin comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 2: The medical device of aspect 1, wherein the header is formed from a polyetherimide resin comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 3: The medical device of any one of aspects 1-2, wherein the enclosure further comprises an antenna.

Aspect 4: The medical device of any one of aspects 1-3, wherein the polyetherimide has a molecular weight of at least 40,000 Daltons.

Aspect 5: The medical device of any one of aspects 1-3, wherein the polyetherimide has less than 100 ppm amine end groups and a weight average molecular weight of 10,000 to 80,000 Daltons.

Aspect 6: The medical device of any one of aspects 1-5, wherein the polyetherimide resin is a fiber polymer having a diameter of fibers of from about 0.00001 millimeters to about 2 millimeters.

Aspect 7: The medical device of any one of aspects 1-6, wherein the polyetherimide further comprises a biocide or antimicrobial agent, wherein the biocide is selected from germicides, antimicrobials, antibiotics, antibacterials, antiyeasts, antialgals, antivirals, antifungals, antiprotozoals, antiparasites, and combinations thereof.

Aspect 8: The medical device of any one of aspects 1-7, wherein the header is formed from a polymer component comprising between 40 wt % and 90 wt % of the polyetherimide resin and between 10 wt % and 60 wt % of a filler by weight of the polymer component.

Aspect 9: The medical device of aspect 8, wherein the filler comprises glass, carbon, carbon fiber, or a combination thereof.

Aspect 10: A medical device comprising: a hermetically sealed enclosure having one or more spacers disposed within the enclosure, wherein the one or more spacers comprise a polyetherimide; and a header coupled to the enclosure to provide electrical communication to an element within the enclosure.

Aspect 11: The medical device of aspect 10, wherein the polyetherimide resin comprises structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 12: The medical device of any one of aspects 10-11, wherein the polyetherimide has a molecular weight of at least 40,000 Daltons.

Aspect 13: The medical device of any one of aspects 10-11, wherein the polyetherimide has less than 100 ppm amine end groups and a weight average molecular weight of 10,000 to 80,000 Daltons.

Aspect 14: The medical device of any one of aspects 10-13, wherein the polyetherimide resin is a fiber polymer having a diameter of fibers of from about 0.00001 millimeters to about 2 millimeters.

Aspect 15: The medical device of any one of aspects 10-14, wherein the polyetherimide further comprises a biocide or antimicrobial agent, wherein the biocide is selected from germicides, antimicrobials, antibiotics, antibacterials, antiyeasts, antialgals, antivirals, antifungals, antiprotozoals, antiparasites, and combinations thereof.

Aspect 16: The medical device of any one of aspects 10-15, wherein the header is formed from a polymer component comprising between 40 wt % and 90 wt % of the polyetherimide resin and between 10 wt % and 60 wt % of a filler by weight of the polymer component.

Aspect 17: The medical device of aspect 16, wherein the filler comprises glass, carbon, carbon fiber, or a combination thereof.

Aspect 18: A medical device comprising: a header comprising one or more bores; one or more leads disposed in the header and exiting the header through the one or more bores; an enclosure coupled to the header, the enclosure comprising circuitry, a power source, and one or more spacers, wherein the one or more spacers are formed from a polyetherimide resin.

Aspect 19: The medical device of aspect 18, wherein one or more of the header and the spacers is formed from a polyetherimide resin comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 20: The medical device of any one of aspects 18-19, wherein the polyetherimide has less than 100 ppm amine end groups and a weight average molecular weight of 10,000 to 80,000 Daltons.

It is also to be understood that the terminology used herein is for the purpose of describing particular aspects only and is not intended to be limiting. As used in the specification and in the claims, the term “comprising” may include the aspects “consisting of” and “consisting essentially of.” Unless defined otherwise, all technical and scientific terms used herein have the same meaning as commonly understood by one of ordinary skill in the art to which this disclosure belongs.

As used in the specification and the appended claims, the singular forms “a,” “an” and “the” include plural referents unless the context clearly dictates otherwise. Thus, for example, reference to “a polycarbonate” includes mixtures of two or more such polycarbonates. Furthermore, for example, reference to a filler includes mixtures of two or more such fillers.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. A value modified by a term or terms, such as “about” and “substantially,” is intended to include the degree of error associated with measurement of the particular quantity based upon the equipment available at the time of filing this application. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” mean that the subsequently described event, condition, component, or circumstance may or may not occur, and that the description includes instances where said event or circumstance occurs and instances where it does not.

Disclosed are component materials to be used to prepare disclosed compositions as well as the compositions themselves to be used within methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

References in the specification and concluding claims to parts by weight, of a particular element or component in a composition or article denotes the weight relationship between the element or component and any other elements or components in the composition or article for which a part by weight is expressed. Thus, in a composition containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Compounds disclosed herein are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, —CHO is attached through carbon of the carbonyl group. Unless defined otherwise, technical and scientific terms used herein have the same meaning as is commonly understood by one of skill in the art to which this disclosure belongs.

As used herein, the terms “number average molecular weight” or “Mn” can be used interchangeably, and refer to the statistical average molecular weight of all the polymer chains in the sample and is defined by the formula:

Mₙ=ΣNᵢMᵢ/Nᵢ,

where Mᵢ is the molecular weight of a chain and Nᵢ is the number of chains of that molecular weight. Mₙ can be determined for polymers, such as polycarbonate polymers or polycarbonate-polysiloxane copolymers, by methods well known to a person having ordinary skill in the art.

As used herein, the terms “weight average molecular weight” or “Mw” can be used interchangeably, and are defined by the formula:

M_{w}=NᵢMᵢ²/ΣNᵢMᵢ,

where Mi is the molecular weight of a chain and Ni is the number of chains of that molecular weight. Compared to Mn, Mw takes into account the molecular weight of a given chain in determining contributions to the molecular weight average. Thus, the greater the molecular weight of a given chain, the more the chain contributes to the Mw. It is to be understood that as used herein, Mw is measured by gel permeation chromatography. In some cases, Mw can be measured by gel permeation chromatography and calibrated with polycarbonate standards. As an example, a polycarbonate of the present disclosure can have a weight average molecular weight of greater than about 5,000 Daltons based on PS standards. As a further example, the polycarbonate can have an Mw of from about 20,000 to about 100,000 Daltons.

1. A medical device comprising:
a header comprising one or more bores;
one or more leads disposed in the header and exiting the header through the one or more bores;
an enclosure coupled to the header, the enclosure comprising circuitry, a power source, and one or more spacers, wherein the one or more spacers are formed from a polyetherimide resin comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

2. The medical device of claim 1, wherein the header is formed from a polyetherimide resin comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

3. The medical device of claim 1, wherein the enclosure further comprises an antenna.

4. The medical device of claim 1, wherein the polyetherimide has a molecular weight of at least 40,000 Daltons.

5. The medical device of claim 1, wherein the polyetherimide has less than 100 ppm amine end groups and a weight average molecular weight of 10,000 to 80,000 Daltons.

6. The medical device of claim 1, wherein the polyetherimide resin is a fiber polymer having a diameter of fibers of from about 0.00001 millimeters to about 2 millimeters.

7. The medical device of claim 1, wherein the polyetherimide further comprises a biocide or antimicrobial agent, wherein the biocide is selected from germicides, antimicrobials, antibiotics, antibacterials, antiyeasts, antialgals, antivirals, antifungals, antiprotozoals, antiparasites, and combinations thereof.

8. The medical device of claim 1, wherein the one or more spacers are formed from a polymer component comprising between 40 wt % and 90 wt % of the polyetherimide resin and between 10 wt % and 60 wt % of a filler by weight of the polymer component.

9. The medical device of claim 8, wherein the filler comprises glass, carbon, carbon fiber, or a combination thereof.

10. A medical device comprising:
a hermetically sealed enclosure having one or more spacers disposed within the enclosure, wherein the one or more spacers comprise a polyetherimide; and
a header coupled to the enclosure to provide electrical communication to an element within the enclosure.

11. The medical device of claim 10, wherein the polyetherimide resin comprises structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

12. The medical device of claim 10, wherein the polyetherimide has a molecular weight of at least 40,000 Daltons.

13. The medical device of claim 10, wherein the polyetherimide has less than 100 ppm amine end groups and a weight average molecular weight of 10,000 to 80,000 Daltons.

14. The medical device of claim 10, wherein the polyetherimide resin is a fiber polymer having a diameter of fibers of from about 0.00001 millimeters to about 2 millimeters.

15. The medical device of claim 10, wherein the polyetherimide further comprises a biocide or antimicrobial agent, wherein the biocide is selected from germicides, antimicrobials, antibiotics, antibacterials, antiyeasts, antialgals, antivirals, antifungals, antiprotozoals, antiparasites, and combinations thereof.

16. The medical device of claim 10, wherein the one or more spacers are formed from a polymer component comprising between 40 wt % and 90 wt % of the polyetherimide resin and between 10 wt % and 60 wt % of a filler by weight of the polymer component.

17. The medical device of claim 16, wherein the filler comprises glass, carbon, carbon fiber, or a combination thereof.

18. A medical device comprising:
a header comprising one or more bores;
one or more leads disposed in the header and exiting the header through the one or more bores;
an enclosure coupled to the header, the enclosure comprising circuitry, a power source, and one or more spacers, wherein the one or more spacers are formed from a polyetherimide resin.

19. The medical device of claim 18, wherein one or more of the header and the spacers is formed from a polyetherimide resin comprising structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

20. The medical device of claim 18, wherein the polyetherimide has less than 100 ppm amine end groups and a weight average molecular weight of 10,000 to 80,000 Daltons.
